(19) United States
(12) Patent Application Publication
(10) Pub. No.: US 2026/0213000 A1
(43) Pub. Date: Jul. 23, 2026

(54) SYSTEM FOR MANAGING ELECTRONIC CALENDARING SYSTEM OF A FACILITY

(71) Applicant: OTTAWA HEART INSTITUTE RESEARCH CORPORATION, Ottawa (CA)

(72) Inventor: Louise SUN, Ottawa (CA)

(21) Appl. No.: 19/327,341

(22) Filed: Sep. 12, 2025

Related U.S. Application Data
(63) Continuation-in-part of application No. 17/912,887, filed on Jul. 23, 2021, now abandoned.
(60) Provisional application No. 63/055,620, filed on Jul. 23, 2020.

(51) Int. Cl. G16H 40/20; G06Q 10/1093

(52) U.S. Cl. CPC G16H 40/20; G06Q 10/1093

(57) ABSTRACT

System for managing an electronic calendaring system for a facility, such as a healthcare facility. The system comprises a server that receives patient data from a source. The patient data (and/or other data) is then fed into a Length-of-Stay (LOS) model and a separate waitlist model. Outputs of the LOS model and the waitlist model are then passed to a scheduling model. The scheduling model preferably comprises a trained AI model that outputs one or more schedule elements configured for entry in an electronic calendaring system. Each schedule element has a set of components that uniquely designate a specific event, when taken in combination. The components are automatically determined by the scheduling model, and may include at least a type of a medical procedure; a date, time, and location for said medical procedure; and lists of resources and invitees for said medical procedure.

### CROSS-REFERENCE TO RELATED APPLICATIONS

This application is a Continuation-in-Part application that claims priority to and the benefit of the non-provisional U.S. patent application Ser. No. 17/912,887, titled “HEALTHCARE RESOURCES MANAGEMENT”, which itself entered the US National Stage under 35 U.S.C. § 371 on Sep. 20, 2022, claiming the benefit of international application number PCT/CA2021/051033, which was filed in the World Intellectual Property Org. (WIPO) on Jul. 23, 2021, and of U.S. Provisional Patent Application No. 63/055,620, filed in the United States Patent and Trademark Office on Jul. 23, 2020. The specifications of the above referenced patent applications are incorporated herein by reference in their entirety.

### TECHNICAL FIELD

The present invention relates to facility management. More specifically, the present invention relates to systems and methods for managing an electronic calendaring system of a healthcare facility such as a hospital.

### BACKGROUND

Healthcare resource management at a healthcare facility includes, among other things, the coordination of personnel and the triaging of care. These is an ongoing challenge for healthcare systems, and especially publicly funded healthcare systems, because available resources are finite. This challenge has become global since the onset of the coronavirus disease (COVID-19) pandemic, as many non-emergent procedures were postponed to preserve system capacity for patients with COVID-19. The recent COVID-19 global health crisis caused elective surgeries to be postponed to limit infectious exposure and to preserve hospital capacity. However, this ramp down in elective surgery volumes may result in unintended harm to patients who are at high risk of mortality if their conditions are left untreated.

By the time that COVID-19 was declared a pandemic by the World Health Organization (WHO), the availability of intensive care unit (ICU) resources had already begun to fall short of the increasing number of critically ill patients in some regions. Amidst this crisis, however, surgical patients continued to require life-saving ICU resources. For example, a significant number of patients with advanced, symptomatic cardiac diseases continue to require cardiac surgery on an urgent basis to prevent disease decompensation and death. This need challenged system capacity, given the complex comorbidities that often co-exist with cardiac surgical disease, as well as the demand for ICU monitoring following major surgeries.

The current paradigm of triage decision-making is primarily driven by clinicians' judgment and experience, which has been shown to be highly inaccurate in predicting prolonged cardiac surgical ICU (CSICU) length of stay (LOS). Although several objective clinical CSICU LOS models have been proposed, they are all built upon small single-center datasets, lack multicenter external validation, and rely on intra- and postoperative data to achieve modest discrimination. With a goal to save more lives while maintaining an efficient and adaptable allocation of critical care resources, there is a need for better methods for managing such scarce resources such as ICU capacity. The experience of the COVID-19 pandemic has led to a need for better procedures.

Any suitable management system for ICU and other scarce resources (such as surgical assets) should take into account the waitlists for current patients, as well as those patients coming into the healthcare pipeline. To date, most studies of waitlist mortality have been centered on major noncardiac surgery and/or cardiac transplantation. A study by an Alberta-based group that investigated 101 cardiac waitlist deaths found that adherence to Canadian Cardiovascular Society (CCS) waitlist recommendations poorly predicted cardiac surgical waitlist mortality (c-statistic 0.577) and many patients died within recommend waitlist timeframes. The poor ability of the CCS waitlist recommendations to prevent deaths suggests a need to re-evaluate cardiac surgery triage criteria, without relying on clinicians' instinctive judgments.

Accordingly, there is a need for systems and methods for managing scarce healthcare resources. Preferably, such systems and methods should address waitlists, mortality rates of those on the waitlists, and the length of stay in critical wards for patients, while minimizing facility occupancy rates and increasing facility throughput. Even more preferably, such systems and methods should be simple to use and provide acceptable levels of accuracy, to enable facilities to provide suitable care when needed.

### SUMMARY

The present invention provides a system for managing an electronic calendaring system for a facility, such as a healthcare facility. The system comprises a server that receives patient data from a source. The patient data (and/or other data) is then fed into a Length-of-Stay (LOS) model and a separate waitlist model. Outputs of the LOS model and the waitlist model are then passed to a scheduling model. The scheduling model preferably comprises a trained AI model that outputs one or more schedule elements configured for entry in an electronic calendaring system. Each schedule element has a set of components that uniquely designate a specific event, when taken in combination. The components are automatically determined by the scheduling model, and may include at least a type of a medical procedure; a date, time, and location for said medical procedure; and lists of resources and invitees for said medical procedure.

In a first aspect, this document discloses system for managing an electronic calendaring system, said system comprising:
a server configured to produce at least one schedule element detailing at least one medical procedure for a specific patient at a healthcare facility, and said server being configured to:
   receive patient data, said patient data relating to said specific patient;
   implement a length of stay (LOS) model, said LOS model producing at least one LOS probability related to said specific patient's projected LOS at said healthcare facility, wherein said at least one LOS probability is based on said patient data;
   implement a waitlist model for calculating a waitlist probability based on said patient data, said waitlist probability being a probability of an emergent event of said specific patient while said specific patient remains on a waiting list for said at least one medical procedure;
   implement a scheduling model, said scheduling model being a trained AI model configured to receive said at least one LOS probability, said waitlist probability, and current occupancy data of said healthcare facility as inputs, and said scheduling model being configured to output said at least one schedule element;
   receive said at least one schedule element as output from said scheduling model; and
   automatically enter said schedule element in said calendaring system,
wherein said at least one schedule element is configured for entry in said electronic calendaring system,
wherein said at least one schedule element has a specific set of components,
wherein said components of said schedule element comprise: a type of a medical procedure; a date for said medical procedure; a time for said medical procedure; a location for said medical procedure; a list of resources required for said medical procedure; and a list of invitees for said medical procedure, and
wherein values of said components are automatically determined by said scheduling model.

In another embodiment, this document discloses a system wherein said scheduling model predicts a cumulative distribution of occupancy at said healthcare facility over a predetermined time period.

In another embodiment, this document discloses a system wherein said scheduling model minimizes said cumulative distribution when determining said values of said components.

In another embodiment, this document discloses a system wherein entering said schedule element in said calendaring system causes at least one further change in said calendaring system, and wherein specifics of said further change are also determined by said scheduling model.

In another embodiment, this document discloses a system wherein said server is further configured to inspect said schedule element before entering said schedule element in said calendaring system, wherein inspection of said schedule element comprises comparing values of said components of said schedule element to values of components of other calendar entries already in said calendaring system, such that, when values of a subset of said components of said schedule element match values of corresponding components of a specific entry of said other entries, said server identifies a conflict between said schedule element and said specific entry.

In another embodiment, this document discloses a system wherein, when said conflict is identified, said server feeds back said at least one LOS probability and said waitlist probability of said specific patient to said scheduling model, along with input data for said specific entry, to thereby produce at least one of a revised schedule element and a revised specific entry, said revised schedule element having at least one component value that differs from a component value of said schedule element and said revised specific entry having at least one component value that differs from a component value of said specific entry.

In another embodiment, this document discloses a system wherein said scheduling model produces both a revised schedule element and a revised specific entry.

In another embodiment, this document discloses a system wherein said scheduling model also takes existing entries in said calendaring system as inputs, and wherein said values of components of said schedule element do not conflict with components of said existing entries.

In another embodiment, this document discloses a system wherein said scheduling model generates a schedule element for all patients at said healthcare facility at a specific time, thereby generating a full schedule for medical procedures at said healthcare facility.

In another embodiment, this document discloses a system wherein said scheduling model is trained on population-wide data and on historical data from said healthcare facility.

In another embodiment, this document discloses a system wherein said emergent event is at least one of an unplanned hospitalization of said at least one patient and a mortality of said at least one patient.

In another embodiment, this document discloses a system wherein at least one of said LOS model and said waitlist model is a trained AI model.

In another embodiment, this document discloses a system wherein both of said LOS model and said waitlist model are trained AI models.

In another embodiment, this document discloses a system wherein said list of invitees comprises at least one medical professional.

In another embodiment, this document discloses a system wherein said at least one medical professional is identified by said scheduling model by assessment of said type of said medical procedure and of personnel records of said healthcare facility, such that said at least one medical professional has a suitable skill level for said medical procedure.

In another embodiment, this document discloses a system wherein said server is further configured to transmit said schedule element to at least one person on said list of invitees before entering said schedule element in said calendaring system.

In another embodiment, this document discloses a system wherein said cumulative distribution of occupancy at said healthcare facility is a predicted distribution of occupancy of a specific subunit of said healthcare facility.

In another embodiment, this document discloses a system wherein said specific subunit of said healthcare facility is an Intensive Care Unit (ICU).

In another embodiment, this document discloses a system wherein said cumulative distribution of occupancy at said healthcare facility comprises a predicted distribution of occupancy in an Intensive Care Unit (ICU) and a predicted distribution of occupancy in said healthcare facility.

In another embodiment, this document discloses a system wherein said at least one patient is designated a high-risk patient when said waitlist probability of said at least one patient is high and wherein said scheduling model is trained to prioritize medical procedures such that high-risk patients are scheduled for near-term medical procedures.

### BRIEF DESCRIPTION OF THE DRAWINGS

The embodiments of the present invention will now be described by reference to the following figures, in which identical reference numerals in different figures indicate identical elements and in which:

FIG. 1 is a block diagram illustrating a system according to one aspect of the present invention;

FIG. 2 is a screenshot of an application that uses the system of FIG. 1;

FIG. 3 is a screenshot of a data input screen that shows the application can simultaneously ingest data for multiple patients;

FIG. 4 is a screenshot of a data entry screen for scheduling a patient's surgery; and

FIG. 5 is a screenshot of a data entry screen for entry of data for a specific patient.

### DETAILED DESCRIPTION

The present invention provides a system for managing an electronic calendaring system for a facility, such as a healthcare facility. As detailed above, conventional methods are ill-suited to optimizing the scheduling of treatment (i.e., surgeries and other medical procedures) for multiple patients in busy facilities. Thus, the present system provides automated/autonomous treatment scheduling based on data-driven predictions of patient needs. The system predicts the likely Length-of-Stay (LOS) of a patient at the facility, i.e., the time that the patient will need to remain at the facility to recover following a medical procedure. The system also predicts that patient's “waitlist probability”, i.e., the likelihood that the patient will suffer an “emergent event”, such as mortality, while remaining on a waitlist for a medical procedure. The LOS and the waitlist probability are predicted based on patient data, including details about the patient in question and also on population data/demographic data, etc. Then, based on the predicted LOS and the waitlist probability, as well as on data from the healthcare facility (such as, without limitation, bed and resource availability and personnel data), the system automatically schedules at least one medical procedure (e.g., a surgery) for the patient. The system can then automatically enter that procedure in an electronic calendar/calendaring system used by the facility. The scheduling may be performed using one or more trained AI models, depending on the embodiment. Further, the system, in some embodiments, adjusts or moves calendar entries for other procedures and/or other patients to accommodate a newly scheduled procedure.

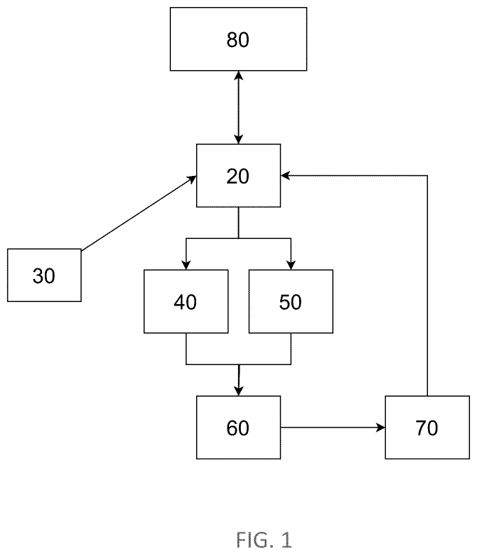

Referring to FIG. 1, a block diagram of a system according to one aspect of the invention is illustrated. As can be seen, the system 10 comprises a server 20 that receives patient data from a source 30. The patient data is then fed into a Length-of-Stay (LOS) model 40 that is implemented by the server 20, and fed into a separate waitlist model 50 that is implemented by the server 20. As detailed below, both the LOS model 40 and the waitlist model 50 may take other relevant data as inputs. Outputs of the LOS model 40 and the waitlist model 50, along with additional data described below, are then passed to a scheduling model 60. The scheduling model 60 preferably comprises a trained AI model that outputs one or more schedule elements 70. Each schedule element 70 has a set of components that uniquely designate a specific event, when taken in combination. The components are automatically determined by the scheduling model 60. The at least one schedule element 70 is configured for entry in an electronic calendaring system 80. The at least one schedule element 70 is received by the server 20 which passes the schedule element(s) 70 to the electronic calendaring system 80 for entry.

In one embodiment, the components of each schedule element comprise: a type of a medical procedure; a date for the medical procedure; a time for the medical procedure; a location for the medical procedure; a list of resources required for the medical procedure; and a list of invitees for the medical procedure. As should be understood, the list of invitees typically comprises a patient and at least one medical professional. The list of resources may comprise specific tools, technologies, or equipment (e.g., anesthetizing equipment, imaging machines, etc), as well as specific medications, medicaments, and/or assistive devices.

As should be understood, some schedule elements may share specific values for these components. For example, multiple medical procedures of the same type may occur at the same time. Similarly, a patient (one of the invitees) may require a number of medical procedures that all occur in the same location (e.g., a specific operating room). As another example, a surgeon (another potential invitee) may perform several procedures of the same type on the same day. However, as should also be understood, different events designated by schedule elements may conflict with each other. A conflict may be identified by comparing components of two schedule elements (or of one newly generated schedule element and a preexisting calendar entry). When multiple components match, a conflict is identified. As an example, a single surgeon may perform multiple medical procedures of the same type (for different patients) on the same day and in the same location. However, that surgeon cannot perform all of those medical procedures at the same time. Similarly, a patient cannot be scheduled for two simultaneous procedures in different locations with different medical teams in attendance.

It is also possible that an identified conflict may not be a true conflict. For example, a medical professional may perform a specific procedure a number of times, for a number of patients, in a single location at a specific time. An example of such a false conflict may be a mass vaccination procedure, in which a number of patients sign up to be seen during a time slot (e.g., ‘between 9 am and 12 μm on November 11 at the community centre’).

Thus, in some embodiments, the server 20 inspects each schedule element 70 before entering that schedule element 70 in an electronic calendaring system to determine whether the new element conflicts with any existing elements. The server 20 is also preferably configured to access and process contextual information regarding the nature/type of the procedure. Such contextual information may be generated by the scheduling model 60. The contextual information may also be included in metadata of the schedule element 70, may be retrieved from the data source 30, and/or may be presubmitted by a user of the system 10, depending on the embodiment.

In some embodiments, when the server 20 identifies a conflict between a new schedule element and an existing calendar entry, either or both of that new schedule element and the existing entry are passed back to the scheduling model 60. The relevant LOS and/or waitlist probabilities may also be reinput to the scheduling model 60. These relevant LOS and/or waitlist probabilities may include the probabilities that were used by the scheduling model as inputs when generating the schedule element and other entry. As well, the scheduling model 60 may receive other input data such as an indication of the nature of the conflict. The scheduling model 60 then outputs either a revised schedule element or a revised other entry, or both. As should be understood, the values of the specific conflicting components of the revised schedule element and/or the revised other entry are modified from the original values of the original schedule element and/or other entry to thereby avoid the conflict.

As should be clear, when a schedule element 70 conflicts with another calendar entry, the revised schedule element 70 and/or revised entry may themselves conflict with still other entries. Accordingly, the scheduling model 60 may continue to revise entries until no conflicts in the calendaring system are found. However, depending on the embodiment, it may be preferable for the scheduling model 60 to generate a full schedule (i.e., one or more calendar files comprising data on a plurality of individual events) each time a new event/procedure is to be added to the schedule. This approach would prevent lengthy conflict-resolution processes in which each revision creates a new conflict that must itself be resolved. However, as may also be imagined, a constantly changing schedule may be difficult to practically implement or follow. Thus, the scheduling model 60 may also be configured to follow one or more logic rules. For example, the scheduling model 60 may be prevented from changing a specific calendar entry if more than four changes have already been made to that specific calendar entry. As another example, the scheduling model 60 may be prevented from changing a specific calendar entry if its time slot begins in under 1 hour.

In some embodiments, each schedule change is accompanied by real-time patient status updates that are sent to at least some of the list of invitees. The server 20 can be configured to send push notifications to relevant medical professionals with each scheduling change. Additionally, the server 20 can be configured to automatically send such notifications to one or more authorized third parties, such as holders of medical powers of attorney, approved family members, parents/guardians, etc.

The schedule element(s) 70 comprise electronic files/computer files that are compatible with electronic calendaring system such as Microsoft Outlook™, Google Calendar™, Apple Calendar™, eM Client™, HCL Domino™, Mozilla Thunderbird™, and other similar systems. For example, the schedule element(s) may be files having .ICS (iCalendar), .PST, or .OST file formats, and/or other suitable file formats. In some embodiments, each schedule element may be provided in multiple formats simultaneously. Additionally, in some embodiments, a user may instruct the scheduling model 60 to provide the schedule element(s) in a specific format. As well, in some embodiments, the scheduling model may generate schedule elements in one specific format that may then be converted into a different format. Thus, in some embodiments, the system 10 may include a conversion utility for converting calendar files to other formats. The format of the schedule element preferably comprises a known and regular list of components that may be easily populated with specific values by the scheduling model 60.

In preferable embodiments, the electronic calendaring system 80 comprises a calendaring system used by the healthcare facility. For example, the calendaring system may be a global calendaring system used by the entire healthcare facility or a calendaring system used by a specific division, department, floor, unit, or ward (or other similar subunit of the healthcare facility). In such embodiments, the server 20 preferably has direct access to the calendaring system, such that the server 20 can add, modify, and/or delete entries in the calendaring system without human intervention. The server 20 may also, in some embodiments, send the schedule element 70 to an individual's private calendaring system (e.g., a patient's private/personal calendar, which the patient manages using their own personal electronic device(s)). In such embodiments, the server 20 provides the schedule element 70 to the patient's device(s) in a downloadable electronic format.

In some embodiments, the scheduling model 60 is configured to minimize an occupancy of said healthcare facility. The occupancy or occupancy rate may be understood as the number of beds occupied at any time (i.e., the number of patients needing treatment). Although the term ‘beds’ is used herein, it should be understood that outpatient facilities and other facilities may also benefit from the scheduling model of the present disclosure. That is, reducing the pendency of patients' visits and the number of emergent events experienced by patients who are waiting for treatment may be of interest for numerous different healthcare facilities. Such different healthcare facilities may include day clinics and general practitioners. Such different healthcare facilities may also include specialists, inpatient facilities, hospitals, and long-term treatment facilities. As should be clear, the use of the term ‘beds’ herein should not be considered to limit the scope of the invention in any way.

The occupancy rate may be modeled as a function of various parameters in the healthcare facility. Additionally, depending on the embodiment, the occupancy rate may be predicted and/or determined by the scheduling model 60 based on its input data. The occupancy rate may be a cumulative distribution function (CDF) for occupancy over time and/or may be a specific value or predicted value corresponding to a specific time. In some embodiments, the scheduling model 60 is configured to predict a cumulative distribution of occupancy for the healthcare facility and to determine schedule element(s) that satisfy a minimum (or one or more local minima) of the occupancy distribution function. Depending on the embodiment, the scheduling model 60 may also provide the predicted/generated occupancy distribution function to one or more users as an output, in addition to the schedule elements.

It should be understood that a Cumulative Distribution Function (CDF) of occupancy function Occ is a probability distribution representing the likelihood that the occupancy function, evaluated at any point x, will be less than or equal to x, where x is the maximum occupancy of the healthcare facility (or of the subunit, e.g., unit, department, floor, ward, etc.) That is:

$${F}_{Occ} ( x ) = P ( Occ \leq x )$$

The scheduling model 60 is preferably provided with as much training data as possible. For example, the scheduling model 60 is preferably trained on historical data from the specific healthcare facility, as well as on data from other similar healthcare facilities and population-wide data. The scheduling model 60 preferably receives existing and historical schedules, outcomes (including mortalities), length-of-stay data, information on waitlist progress and outcomes (including emergent events experienced by waitlisted patients), demographic information for the patients, information on comorbidities of the patients, and/or other patient information to use as training data. As well, the scheduling model 60 is preferably trained on personnel data and other local data for the healthcare facility (i.e., data related to the medical professionals, infrastructure, and equipment available to the specific healthcare facility where the system 10 is implemented). After training, the scheduling model 60 may obtain current local data for the healthcare facility, including personnel and other administrative information of the healthcare facility, on an ongoing basis. For example, the scheduling model 60 may retrieve such data from one or more databases of the healthcare facility at the beginning of each week or month. Such data may include human-forecasted limitations and predictions, such as the type and number of procedures anticipated, the daily availability of surgeons, anesthesiologists, assistants, perfusionists, nurses, etc., planned infrastructure, construction, or maintenance work, and other known data.

In one variant of the system described above, any changes, optimizations, or edits to schedules made by the system are sent to a human for validation/confirmation. Thus, any scheduling decisions made by the system are first reviewed/validated by a human before being finalized. Such a human reviewer can, when necessary, override the scheduling decisions made by the system. In the event of such an override, the system may need to rework the schedule to take into account the human override.

The reworked schedule will, of course, require human approval and verification before being finalized and implemented. As noted above, the scheduling and resource management may include operating room (OR) scheduling, surgical team scheduling, nurse/care worker scheduling, surgical procedure scheduling, relevant work assignments, as well as other management functions that can take into account patient care/condition.

Additionally, in some embodiments, the scheduling model 60 may comprise one or more logic rules that operate as limits or constraints on the model 60. These logic rules are not derived from the model's training data. For example, the scheduling model 60 may be initially configured with rules such as ‘no major surgeries on weekends’ or ‘high-mortality-risk patients are to be prioritized above all others’. These rules may be permanently applied by the scheduling model 60 or may be subject to modification by the scheduling model 60. In addition, some rules may conflict with other rules. For example, if a patient is triaged on a Friday night and has a 95% waitlist probability over the next two days (i.e., a 95% chance of mortality before Monday without treatment), a rule against weekend procedures may be overruled. Of course, as should be understood, such rules and any relationships between such rules may be highly localized/unique to the specific healthcare facility where they are implemented.

Additionally, in some embodiments, the scheduling model 60 may rearrange all cases/procedures/schedules at the healthcare facility, rather than responding to one or more specific incoming patients. Similarly, the scheduling model 60 may rearrange any subset of cases/procedures/schedules at the healthcare facility, and/or cases/procedures/schedules corresponding to specific units, sections, subunits, etc. of the healthcare facility. This rearranging may be referred to as “scrambling” existing schedule information to arrive at a new schedule. The new schedule is preferably optimized to provide one or more desired advantages over the original schedule (e.g., to further minimize occupancy, to account for other scheduling needs at the healthcare facility, to prioritize a specific procedure or a specific type of procedure, etc.). In such implementations, the scheduling model 60 may receive input data only related to existing patients with existing scheduled procedures, rather than data related to newly arrived patients or waitlisted patients.

It should also be understood that the healthcare facility may be the entire facility, such as a hospital, or may be a specific subunit of a facility. For example, the scheduling model 60 may create a schedule exclusively for an Intensive Care Unit (ICU) of a hospital, without reference to other units/subunits of the hospital. However, as the ICU may share resources and/or operating room space with the rest of the hospital, such siloed scheduling may not be preferable. Accordingly, in some embodiments, multiple schedules are created by the scheduling model, including schedules for units/subunits and an overall schedule for the entire facility. As described above, this may require generating occupancy rate distributions for each subunit of interest and an occupancy rate distribution for the facility as a whole. The occupancy rate distribution for the facility as a whole may, in some embodiments, be based on the distributions for the subunits. For example, if occupancy distributions are determined for all subunits of a healthcare facility, the overall occupancy distribution may be determined by overlaying and/or summing the separate subunit distributions together.

In some embodiments, the scheduling model 60 can be configured to predict and output general staffing needs for a forthcoming period at the healthcare facility. The period may be of any length (e.g., a day, a week, a month, or more). As should be understood, longer periods may be more difficult to predict, as there is more time for deviation from the predicted schedule and a higher likelihood of chaotic events derailing the predicted schedule.

For example, as noted above, if a patient on the waiting list has a high probability of mortality within 2 days, the schedule element 70 produced by the scheduling model 60 would address that high probability of mortality—i.e., would schedule at least one urgent medical procedure for that patient. Similarly, the server 20 may compare two incoming patients (or more) when data for each is received, and generate a forward schedule based partially on their predicted mortalities/hospitalization risk and predicted lengths of stay. As an example, consider incoming patients A and B who both have an 80% chance of requiring 2 days or less in critical care, and incoming patient C, who has a 75% chance of requiring more than 7 days of critical care. If there are currently 7 free spots available in the critical care unit, the server 20 would forecast that, for at least the next 2 days, there will only be 4 available spots in critical care. Similarly, from the data above, the server 20 would forecast that there will be 6 available spots in critical care 3 days from now (i.e., once patients A and B are discharged from critical care).

As another example, the scheduling of surgical procedures may account for the calculated probabilities of mortality for patients on the waiting list. Patient A may have a 40% probability of mortality within the next 3 days while patient B may only have a 10% probability of mortality within the next 3 days. Once a surgical slot opens up, in this example, the schedule elements for patients A and B may thus schedule patient A's procedure before patient B's procedure. It should be noted that the trained AI model(s) that comprise(s) the scheduling model 60 may not demonstrate exactly traceable relationships between incoming LOS and waitlist probabilities. That is, other factors (including without limitation the availability of medical professionals and/or equipment) may also be accounted for by the scheduling model, such that an exact relationship between waitlist or LOS probabilities and the components of the schedule element(s) 70 are difficult to determine retroactively. Depending on the implementation, the lists of invitees on each schedule element 70 may comprise the first available surgical team or the best surgical team for the procedure.

That is, in some embodiments, the scheduling model 60 determines at least a portion of the list of invitees for a specific schedule element 70 by reference to personnel data/personnel records of said healthcare facility. In such embodiments, the medical professional(s) assigned to a specific medical procedure are preferably selected based on both their availability and their skill level(s), with reference to the nature/type of the medical procedure. For example, some complex procedures may require a particular surgeon with a particular speciality. Accordingly, those procedures would need to be scheduled with that particular surgeon and the timing of those procedures would be dependent on that surgeon's availability. On the other hand, less complex procedures may be performed by a larger number of medical professionals, having a wider range of overall skill levels. For such procedures, the specific medical professional(s) included in the list of invitees is less important. In preferable embodiments, the scheduling model 60 is trained/configured to assess the type of medical procedure and identify medical professional(s) who have a suitable skill level to perform the medical procedure within a reasonable time period. Of course, the length of such a “reasonable” time period depends on the nature of the medical procedure, the waitlist and LOS probabilities for the patient, and the occupancy/availability at the healthcare facility. In addition and/or alternatively, the scheduling model 60 may select medical professionals based in part on patient preference and/or administrative decisions of the healthcare facility, provided such information is transmitted to the scheduling model 60.

Additionally, in some embodiments, the server 20 transmits a schedule element 70 to at least one of the people on the list of invitees before entering the schedule element in the calendaring system. For example, the schedule element 70 may be sent to one or more of the listed medical professionals. The medical professional(s) may then determine whether the automatically generated schedule element is acceptable. For example, a particular surgeon may be available at a certain time selected by the schedule element 60, but may have just completed a different, exhausting surgery and require more rest time in between. Thus, in some embodiments, some of the invitees may be provided with the opportunity to reject a scheduled procedure and request re-scheduling. As well, in some embodiments, all invitees may be sent an acceptance request in respect of the schedule element before the server 20 adds the schedule element 70 to the calendaring system.

In some embodiments, the server 20 comprises a single physical server unit. Such a single unit may be at the healthcare facility (i.e., “on prem” or “on-premise”) or may be physically remote from the healthcare facility. In some embodiments, the server 20 comprises multiple physical and/or software units that cooperate under the direction of a primary control unit. In some embodiments, the server 20 comprises a distributed server. The various models implemented by the server may be implemented remotely, in a distributed manner, and/or using circuitry of the single physical server 20, depending on the embodiment.

The data source 30 may comprise one or more databases. As examples, the data source 30 may comprises a patient database and/or a personnel database of the healthcare facility. However, other data may also be retrieved/received from a live data source 30, such as by way of data entry by one or more healthcare professionals (e.g., an attending physician, triage nurse, etc. using a computing device, including without limitation a mobile computing device). Data may be retrieved/provided at separate times for separate patients or in batches, depending on the implementation. Additionally, the retrieved data may relate to a single patient or to multiple patients.

In some embodiments, the LOS model 40 and/or the waitlist model 50 comprise trained AI models that are trained using data on relevant populations. In other embodiments, the waitlist model 50 and/or the LOS model 40 may be configured using a combination of human logic and machine learning elements, or may be configured using only human logic. Possible exemplary implementations of the waitlist model 50 and the LOS model 40 follow.

### Exemplary Implementation of Waitlist Model(s) 50

The waitlist model determines the likelihood of a specific patient experiencing an emergent event while on a waiting list (i.e., while not receiving treatment). Emergent events generally comprise one or more of: unplanned hospitalization, unexpected worsening of a condition, and mortality. Patients with high risks of emergent events may be prioritized over all other patients in a schedule, depending on the implementation. In particular, as further described below, some embodiments of the waitlist model comprise multiple models, such as a model that evaluates a risk of death/mortality and a model that evaluates the risk of hospitalization. Depending on the implementation, high risk patients may be identified as those with high risks of death, while patients with lower risk of death may be considered lower risk patients, notwithstanding a high risk of hospitalization. It should also be understood that ‘high’ and ‘low’ risk may be assessed based on absolute standards. For example, ‘high risk’ may correspond to any waitlist probability above 75%, while ‘low risk’ may correspond to any waitlist probability of 25% or lower. Alternatively, high risk and low risk may be flexible standards. For example, in some embodiments, patients with waitlist probabilities in the highest decile of all current patients may be deemed to be high-risk patients, even if the absolute value of the waitlist probability for those patients is fairly small.

The waitlist model(s) is preferably developed using a large sample data set. From the data set's data, multiple interrelated models were derived and, based on a number of factors, the probability of mortality for patients with specific ailments and conditions was calculated. In one exemplary implementation, a waitlist model was designed specifically to address cardiac patient waitlists. For this implementation, a cohort study of adult patients ≥18 years of age, who were placed on the waitlist for coronary artery bypass grafting (CABG), and/or aortic, mitral, tricuspid valve, or thoracic aortic surgery in Ontario within a specified date window was performed. Excluded were patients who are waitlisted for transcatheter procedures, as well as for cardiac transplantation and ventricular assist devices. As data sources for this study and model extraction, the clinical registry data from the province of Ontario, and population level administrative healthcare databases with information on all Ontario residents was used. Using unique confidential identifiers, the Ontario registry (waitlist management, date and type of procedure, physiologic and comorbidity data) was linked with the Canadian national database for hospital admissions, the Ontario physician service claims database, and the vital statistics database. These databases have been validated for many outcomes, exposures, and comorbidities. For this specific model extraction, outcomes (i.e., including emergent events and the absence of emergent events) were recorded as occurring between referral date and surgery. The primary outcome focused on in this model was mortality/death. The secondary outcome was non-elective hospitalization due to any cause (i.e., both cardiac causes and all other causes). It should be clear that, in one implementation, the waitlist model allows for a user-adjustable time frame over which the patient's probability of death or non-elective hospitalization may be calculated. It should also be clear from the description below that other models focused on the prediction of other outcomes (such as a composite of death and non-elective hospitalization and non-elective hospitalization alone) were also created. Further, it should be noted that the waitlist model may comprise multiple subsidiary and/or interlocking models that assess and predict specific probabilities/outcomes. The discussion regarding such waitlist models follows below, after discussion regarding the model where death is the primary outcome.

Other potential covariates were used in developing the exemplary model, including (but not limited to) age, sex, smoking, hypertension, left ventricular ejection fraction (LVEF), myocardial infarction (MI) within 30 days prior to surgery, CCS angina class, New York Heart Association (NYHA) functional status, atrial fibrillation, heart failure (HF), stroke, endocarditis, peripheral arterial disease, chronic obstructive pulmonary disease, glomerular filtration rate, dialysis dependence, diabetes, anemia, redo sternotomy, type of surgery, and procedure urgency. Additionally, the following anatomic variables were evaluated: number and location of diseased coronary arteries, presence of left main, left main (LM) equivalent and proximal left anterior descending artery (LAD) disease, and the type and severity of valvular lesions. The values for these and other variables may be retrieved by the server 20 from the database for the specific patient being assessed or the values may be retrieved/received from the data source (e.g., an attending physician or some other health professional may enter the values for the variables).

For this derivation (where the primary outcome is death and the secondary outcome is non-elective hospitalization), the cohort was split into a derivation and a validation set by random selection such that 2/3 of the cohort was used to derive the model. The prediction of death was accomplished using a Cox proportional hazards model, while the prediction of non-elective hospitalization using a cause-specific hazard model within a competing risk framework. Variables were included in each of these models if their univariate P-values were <0.25, and retained if they were significant at P<0.05 in the backward elimination model or were deemed a priori to be clinically important. Scores were assigned to each retained covariate based on the method described by the Framingham group. Model calibration was assessed in the validation sample by stratifying patients into risk score strata (using thresholds based on deciles of the risk score determined in the derivation sample) and estimating the incidence of events in each risk stratum. These stratum-specific estimates of risk were compared with mean model-based estimates obtained from the risk score. This risk score was validated using the remaining randomly selected 1/3 of the cohort. (That is, patients with waitlist risks in higher deciles were deemed higher-risk patients.)

It should be clear that different models were developed to determine the probabilities for different outcomes. Again, the model referred to above calculates the probabilities for death as the primary outcome and non-elective hospitalization and the composite of death and non-elective hospitalizations as secondary outcomes. In one variant, a model was developed such that the primary outcome was all-cause mortality that occurred between the date of acceptance onto the waitlist and the date of removal from the waitlist. For this variant, a hybrid approach of Random Forests for initial variable selection was used, followed by stepwise logistic regression for clinical interpretability and parsimony. A bootstrap sample of the data was thus used to build each of the classification trees. A random subset of variables was selected at each split, thereby constructing a large collection of decision trees with controlled variation.

The trees were left unpruned in order to minimize bias. Every tree in the forest casts a “vote” for the best classification for a given observation, and the class receiving the most votes results in the prediction for that specific observation. The dataset was first sampled to create an in-bag partition (2/3 of derivation sample) to construct the decision tree, and a smaller out-of-bag partition (1/3 of derivation sample) was used to test the constructed tree and thereby evaluate its performance. As is known, Random Forests calculate estimates of variable importance for classification using the permutation variable importance measure. This is based on the decrease of classification accuracy when values of a variable in a node of a tree are permuted randomly. This waitlist model variant was based on 500 classification trees and 6 variables available for splitting at each tree node.

For this variant of the waitlist model, a subset of the top 30 predictor variables were identified out of the 40 candidate variables and these were incorporated into a logistic model. Predictor variables were entered into a multivariable backward stepwise logistic regression model based on both clinical and statistical significance, with P<0.10 for entry and P<0.05 for retention. The final prediction model was created and its results can be referred to as a Waitlist Mortality Score. The final model of this variant consisted of 11 variables. These variables included sex, type of surgery, LM-equivalent anatomy, and CCS classification and these variables were forced into the model on the basis of clinical significance. Other multivariable predictors of waitlist mortality were age, LVEF, history of HF, atrial fibrillation, dialysis, psychosis, and operative priority.

In another variant of the present system, a different waitlist model/a variant of the waitlist models above was developed where the primary outcome was the composite of death or unplanned cardiac hospitalization, as defined by non-elective admission for heart failure, myocardial infarction, unstable angina or endocarditis between the date of acceptance and date of removal from the waitlist. For this variant, the cohort was split into a derivation and validation dataset by random selection such that 2/3 of the cohort was used to derive the model. Death or unplanned cardiac hospitalization was predicted using a Cox proportion hazard model. Predictor variables were selected using a backward stepwise algorithm with a significance threshold of P<0.1 for entry and P<0.05 for retention in the model. For continuous variables, their association with the composite outcome was examined using cubic spline analyses with five knots at percentiles 5, 27.5, 50, 72.5 and 95. As there was no violation of the linearity assumption for any of these variables, these were entered into the model as continuous values. This variant model was validated on the remaining 1/3 of the cohort.

For this variant, the predictive model consisted of 16 variables: BMI, acceptance to the waitlist during an inpatient encounter, urban residence, teaching hospital, recent MI within 30 days, CCS and NYHA classification, history of heart failure, atrial fibrillation, diabetes, glomerular filtration rate, proximal LAD disease, aortic stenosis, endocarditis, operative priority at the time of waitlisting, and type of planned surgery.

In terms of implementation, the multiple variants of the different models allow for the waitlist models to calculate different probabilities. The waitlist model(s) can calculate probabilities for: a) mortality alone, b) hospitalization alone, or c) mortality or hospitalization. For mortality alone, two different formulas may be used—the first formula calculates the probability of death as a binary event, irrespective of length of time on the waitlist. The second formula produces time-dependent probabilities of death. As an example, when using the second formula, the probabilities of death at 15, 30, 60 and 90 days after being placed on the waitlist can be calculated. When calculating the probabilities for mortality or hospitalization or hospitalization alone, the waitlist model(s) can be configured to calculate the time-dependent risks for specific time periods. As an example, the waitlist model(s) can calculate the time-dependent probabilities for 15, 30, 60, and 90 days after being placed on the waitlist.

For greater clarity, Tables 4 and 5 are provided below. Table 4 details the baseline characteristics in those who died or had unplanned cardiac hospitalizations and those who did not. Table 5 details the multivariable predictors of death or unplanned cardiac hospitalization while on the waitlist. Note that the data in Tables 4 and 5 relate to cardiac patients.

Again, the waitlist model may comprise multiple models, such that the waitlist probability is a composite value derived from the output of those multiple models. Alternatively, depending on the embodiment, the waitlist model may comprise a suite of models that operate in parallel and that allow users to select an outcome of specific focus. For example, in some embodiments, a user may configure the scheduling model 60 to output a schedule based on the probabilities of waitlisted mortality only. The user may then configure the scheduling model 60 to output a schedule that also accounts for other emergent events (e.g., hospitalization). These selections are preferably made via a user interface.

Again, risk categories and/or risk scores may be developed based on waitlist probabilities (either absolute or relative values, as described above). For some implementations, patients classified as high-risk (in terms of mortality and/or hospitalization) are given precedence/scheduled first for surgical procedures/other treatment and care. Patients classified as having lower risks can be scheduled based on resource optimization methods (e.g., scheduling based on having the optimal surgical team available for higher risk/higher surgical expertise requirements and/or scheduling based on current/projected ICU (intensive care unit) capacity).

It should be clear that, depending on implementation, different pieces of data may be requested as input to the waitlist model. For an exemplary cardiac ward model, different possible inputs may include (without limitation): age, sex, height, weight, the type of hospital the patient is in (teaching hospital, etc.), whether the patient was waitlisted during an inpatient encounter, whether the patient has a rural residence, the CCS (Canadian Cardiovascular Society) classification, whether the patient has had a myocardial infarction within the last 30 days, the New York Heart Association classification for the patient, whether the patient has a history of heart failure, patient conditions and characteristics such as diabetes, proximal LAD, aortic stenosis, LVEF, hypertension, atrial fibrillation, endocarditis, stroke, peripheral arterial disease, anemia, and creatinine readings. As well, the server 20 may request other data about the patient, such as the preoperative cardiogenic shock (or readings that may indicate such), the surgery type the patient requires, and the operative priority for the patient. Any subset of the above may form the input to the waitlist model. As well, other pieces of data may still be requested by the server 20 for the waitlist model(s) depending on implementation.

### Exemplary Implementation of Length Of Stay (LOS) Model(s) 40

The LOS model again may comprise a plurality of subsidiary models. For example, in one exemplary implementation for use in a cardiac ward, two different models were created. Each model predicted whether a given patient was likely to spend a specific amount of time in the critical care unit of the cardiac ward. In this implementation, one of the two models determined the probability that a patient would need less than 2 days of care in the intensive care unit (ICU), while the other model determined the probability that the same patient would need more than 7 days of care in the intensive care unit. These models were derived for cardiac patients as explained below.

However, it should be understood that these models is purely exemplary and that nothing in this section should be considered to limit the scope of the invention. Specifically, the LOS model may comprise any number of models that predict general LOS distributions and/or whether a specific patient is likely to have a stay of a specific length. What follows merely provides context for one implementation of such models.

In one implementation, clinical models were built to predict the likelihood of short (≤2 days) and prolonged ICU LOS (≥7 days) in patients ≥18 years of age was derived and performed. These patients were those who underwent coronary artery bypass grafting and/or aortic, mitral, and tricuspid value surgery in Ontario, Canada. Multivariable logistic regression with backward variable selection was used, along with clinical judgment, in the modeling process. For the model that predicted a short ICU stay (≤2 days), the c-statistic was 0.78 in the derivation cohort and 0.71 in the validation cohort. For the model that predicted a prolonged stay (≥7 days), the c-statistic was 0.85 in the derivation and 0.78 in the validation cohort. The models demonstrated a high degree of accuracy (tested accuracy being greater than 90%) during prospective testing.

For this implementation, an ambispective study was performed, models were derived to predict low and high ICU resource use after cardiac surgery (defined by CSICU LOS of ≤2 and ≥7 days, respectively), using data available at the University of

Ottawa Heart Institute (UOHI). These models were validated using a concurrent multicenter cohort of non-UOHI cardiac surgery patients in Ontario. These models were then tested prospectively at the UOHI.

Inclusion criteria were adult patients ≥18 years of age, who underwent coronary artery bypass grafting (CABG), and/or aortic, mitral, and tricuspid valve surgery. Excluded were patients who underwent procedures requiring circulatory arrest, as well as cardiac transplantation and ventricular assist devices (VAD). For patients with multiple cardiac procedures during the study period, only the index procedure was included in the analyses.

### Derivation Cohort for Short Stay/Long Stay Predictive Model

All 6,625 patients who underwent cardiac surgery at the UOHI within a specific date window and met the selection criteria were included in the derivation cohort. Also used were prospectively collected clinical data from a multimodular data repository that captures detailed demographics, comorbidities, procedural details and outcomes of all patients who underwent cardiac surgical procedures at the UOHI, a university-affiliated tertiary referral center that performs the full scope of cardiac operations.

### Validation Cohort for Short Stay/Long Stay Predictive Model

The validation cohort consisted of cardiac surgical patients from 7 other cardiac care centers in Ontario, who met the selection criteria within a given date window. Also used was the clinical registry data from the province of Ontario, and population level administrative healthcare databases. The clinical registry data from the province of Ontario maintains a detailed prospective registry of all patients who undergo invasive cardiac procedures in Ontario, including demographic, comorbidity, and procedural-related information.

Using unique confidential identifiers, the clinical Ontario registry (that stored the date and type of cardiac procedures, physiologic, and comorbidity data) was linked with the Canadian database for comorbidities and hospital admissions, the provincial database for physician service claims, and the database for vital statistics. These administrative databases have been validated for many outcomes, exposures, and comorbidities, including heart failure, chronic obstructive pulmonary disease, asthma, hypertension, myocardial infarction and diabetes.

Potential covariates considered in the analyses are detailed in Table 1 and included age, sex, body mass index (BMI), smoking, hypertension, left ventricular ejection fraction (LVEF), myocardial infarction within 30 days prior to surgery, Canadian Cardiovascular Society (CCS) angina class, New York Heart Association (NYHA) class, atrial fibrillation, endocarditis, stroke, peripheral arterial disease (PAD), glomerular filtration rate (GFR), dialysis, diabetes treated with oral hypoglycemics and/or insulin, anemia, emergent operative status, preoperative cardiogenic shock, redo sternotomy and type of surgery. The definitions for these variables are provided in Supplemental Table 1 below.

Height and weight were identified from the clinical registry and procedural urgency was ascertained from the clinical registry and database for physician service claims using an established algorithm. In addition, comorbidities were identified from the clinical registry and supplemented with data from the Canadian database for comorbidities and hospital admissions and the provincial database for physician service claims using International Classification of Diseases 10th Revision (ICD-10-CA) codes within five years prior to the index procedure, according to validated algorithms. It should be clear that values for the above noted variables as well as for variables identified below may be retrieved from the database or may be received/retrieved from the data source (e.g. entered by a physician or other healthcare professional).

For this implementation, continuous variables were compared with a 2-sample t-test or with a Wilcoxon rank sum test for non-normally distributed data. Categorical variables were compared with a chi-square test.

In the derivation set, separate logistic regression models were developed to predict the probabilities of CSICU LOS of ≤2 days and ≥7 days, respectively. For each model, univariate logistic regression was used to examine the association of potential predictors that were available at the time of triage and were routinely reported to the clinical registry, with CSICU LOS. According to methods described by others, potential predictors of LOS with univariate P-values of <0.25 were considered for entry into a multivariable logistic regression model based on both clinical and statistical significance. A backward variable selection algorithm was used, retaining in the final multivariable model covariates with P-values of <0.05, as well as those deemed to be clinically important.

Model discrimination in both the derivation and validation datasets was assessed using the c-statistic. Calibration was assessed using the Hosmer-Lemeshow chi-square statistic and by comparing the number of observed vs. expected events in each risk quintile. Model performance was assessed using the Brier score. For each of the LOS models, a predictiveness curve was constructed in the validation dataset by plotting ordered risk percentile on the x-axis, and the probabilities of LOS≤2 days and ≥7 days, respectively, on the y-axis. Other measures of model performance, such as sensitivity, specificity, positive and negative predictive values (PPV, NPV), were determined by examining LOS in higher or lower risk groups at the optimal cutoff value.

These predictive models were tested and descriptive statistics for the testing period are presented below. Analyses were performed using SAS version 9.4 (SAS Institute, Cary, NC), with statistical significance defined by a two-sided P-value of <0.05.

Among the 6,625 patients in the derivation cohort, 4,201 (63.4%) stayed in the CSICU for ≤2 days and 692 (10.4%) for >7 days. Among 65,410 patients in the validation cohort, 50,442 (77.1%) stayed in the CSICU for ≤2 days and 3,364 (5.1%) for ≥7 days. The baseline characteristics of both cohorts were similar, with the exception that patients in the derivation cohort were younger, more likely to undergo complex surgery, to smoke, have atrial fibrillation and anemia. Patients in the validation cohort were more likely to have CCS class 4 symptoms and undergo isolated CABG (Table 1).

The multivariable predictors of short and prolonged CSICU LOS are presented in Table 2. Of the candidate covariates evaluated, younger age, female sex, lower BMI, CCS and NYHA class, higher LVEF, and the absence of atrial fibrillation, endocarditis, stroke, PAD, anemia, higher GFR, emergent operative status, preoperative cardiogenic shock, redo sternotomy, and procedure type, were predictors of short CSICU LOS.

Age and sex were forced into the prolonged LOS model on the basis of clinical significance. Other multivariable predictors of prolonged CSICU LOS were BMI, NYHA class, LVEF, hypertension, atrial fibrillation, endocarditis, anemia, GFR, emergent operative status, preoperative cardiogenic shock, redo sternotomy and procedure type.

For the short stay model, in the derivation dataset, the c-statistic of the multivariable model was 0.78 and the Hosmer-Lemeshow chi-square statistic was 12.71 (P=0.12). In the validation dataset, the c-statistic of the multivariable model was 0.71 and the Hosmer-Lemeshow chi-square statistic was 626.9 (P<0.001). The Brier score was 0.16.

Table 3A shows the observed rates of short CSICU LOS according to each risk quintile. The observed and predicted numbers of patients having LOS≤2 days were similar across all except the lowest probability quintile, where the model tended to underestimate (observed rate 53.4%, predicted 44.3%). On examining a predictiveness curve based on the data, 60% of patients had predicted probabilities exceeding the average rate of short stay. The optimal cutoff point on the ROC curve was at a predicted probability of 76.3%, with the following characteristics: sensitivity, 69.8%; specificity, 60.8%; PPV, 85.7%; NPV, 37.4%.

For the long stay model, in the derivation dataset, the c-statistic of the multivariable model was 0.85 and the Hosmer-Lemeshow chi-square statistic was 18.54 (P=0.02). In the validation dataset, the c-statistic of the multivariable model was 0.78 and the Hosmer-Lemeshow chi-square statistic was 131.43 (P<0.001). The Brier score was 0.047.

Table 3B shows a calibration table showing the rates of prolonged CSICU LOS according to each risk quintile. The number of observed cases having LOS≥7 days was similar to that predicted across all quintiles. Specifically, the average observed probability of short stay was 0.8% in quintile 1 (predicted probability 0.9%), 1.7% in quintile 2 (predicted 1.6%), 3.0% in quintile 3 (predicted 2.5%), 5.5% in quintile 4 (predicted 4.6%), and 14.8% in quintile 5 (predicted probability 17.2%). Based on this data, 22% of patients had predicted probabilities that exceeded the average rate of prolonged stay. The optimal cutoff point on the ROC curve was at a predicted risk of 3.9% (sensitivity, 73.2%; specificity, 68.8%; PPV, 11.3%; NPV, 97.9%). At the 25th, 50th, and 75th percentiles of risk, sensitivities were 95.6%, 85.3%, and 64.1%, respectively, whereas negative predictive values were 99.1%, 98.5%, and 97.5%, respectively.

During a beta testing period for the two LOS models, a total of 42 patients who were evaluated with the models proceeded to have surgery on an urgent basis. Using a predictive threshold of ≥70%, 35 of 38 (92.1%) patients who were predicted to have CSICU LOS of ≤2 days actually did. One patient was predicted to have a LOS of ≥ 7 days but suffered intraoperative death. The remaining three patients were classified as “indeterminate” (i.e., had predicted probabilities of ≤50% for both short and prolonged LOS). Of these patients, two had a LOS of between 2 and 7 days and one ≥7 days.

The two models for LOS may be used to help optimize daily operative planning, whereby scheduling of cases with varying postoperative resource requirements could be staggered to maximize the number of urgent cases performed.

In addition to being fed to the scheduling model, such LOS models may be used to support triaging decisions by complementing the physician's assessment of disease acuity and clinical factors with real-world data. The potential impact of the system depends on the average CSICU LOS durations specific to each institution. At institutions with lower CSICU LOS after cardiac surgery, the system may help to identify the high resource users while, at institutions with longer CSICU LOS, the system may identify those who are likely to have a rapid transition through the CSICU. Given their robust performance in prospective validation, the two LOS models could be used to benchmark the predicted vs. observed CSICU LOS as a quality metric. Such models could also be used to identify patients who may benefit most from preoperative optimization (i.e., those who are mostly to require prolonged LOS).

The LOS model(s) may also comprise, for example, Poisson regression models and/or other models that predict the actual LOS as a continuous variable (e.g., 4.5 days, instead of having a binary cutoff at 2 or 7 days). Additionally or alternatively, the output of multiple LOS models may be combined into a single probability value. Further, in some embodiments, the LOS model(s) output may be a probability that a patient would have up to a predetermined ‘minimum’ length of hospitalization. Conversely, such a model may determine the probability that a patient would have a length of stay that is a predetermined ‘maximum’ LOS.

The projected length of stay and/or probabilities output by the LOS model(s) may be fed to the scheduling model 60 as described above, along with other administrative/local data and waitlist probabilities. The scheduling model 60 would then operate as detailed above.

Again, it should be clear that, while the above descriptions specify cardiac patients as being the subjects for model derivation, models for non-cardiac patients are also possible. The procedure for deriving models for non-cardiac patients would be the same as for cardiac patients but would, of course, involve data for non-cardiac patients. Accordingly, the advantages of the various aspects of the present invention can be extended to include non-cardiac patients.

In terms of implementation, the system may be implemented on a server from which the various models are implemented/operating. The electronic calendaring system 80 may be accessed by users on any number of data processing devices including desktops, laptops, smartphones, and/or other mobile computing devices. The calendaring system may also be integrated into a larger management system that operates/manages a healthcare facility such as a hospital.

In other implementations, the waitlist model and the LOS model may both be implemented in standalone applications that execute/operate either online or on conventional computing devices. Alternatively, the various models of the present invention may be implemented as part of an electronic health record system or as part of a larger system used in or with a health-related facility. As an example, the waitlist model(s) may be resident on a mobile device or may be accessed as an online resource for use by healthcare professionals as necessary. Similarly, the LOS model may be a standalone online or cloud-based resource that may be accessed by healthcare professionals as needed. For these examples, the values for the variables necessary to calculate the relevant probabilities may be entered by one or more healthcare professionals. The resulting probabilities would then be provided to these professionals as standalone numbers for use by the professionals as necessary.

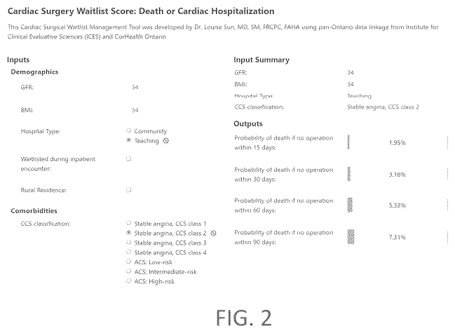

Referring to FIG. 2, a screenshot of an application that uses the models of the present invention is illustrated. The inputs to the application can be seen and these inputs are used to calculate the probabilities relating to one or more specific patients.

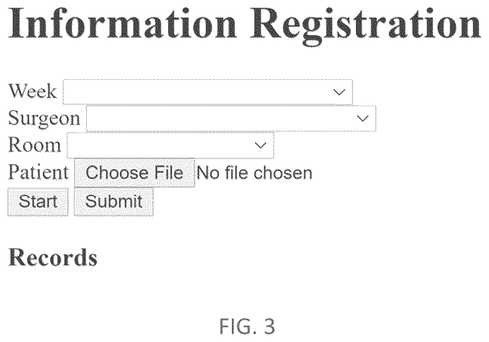

Referring to FIG. 3, another screenshot of data input to the application that uses the above models is illustrated. For this implementation, the system can simultaneously ingest data from multiple patients (by way of a single data file) and can use this data to calculate the probabilities for each patient and to optimally schedule scarce resources based on these probabilities.

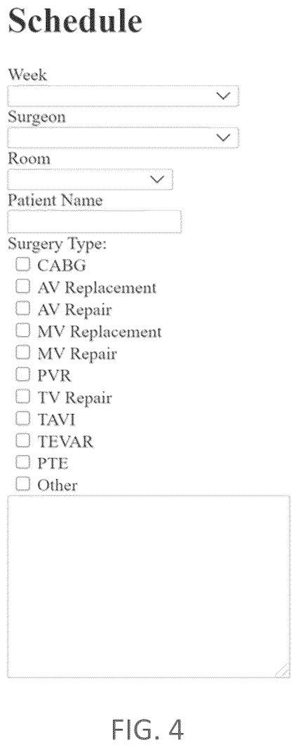

As part of a scheduling application, FIG. 4 is a screenshot of data necessary to schedule an individual patient for surgery. As can be seen, the desired week, surgeon, room, and patient is entered along with the type of surgery. Based on these inputs, the application can optimally schedule the surgery based on the probabilities calculated for this patient and other patients who are similarly waiting for surgery. Similar screens may be used to schedule other scarce hospital resources as necessary.

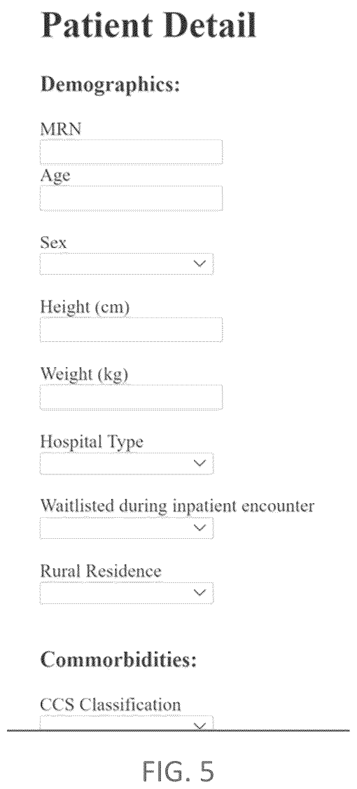

Referring to FIG. 5, a portion of a data entry screen is illustrated for the entry of data for a specific patient. The data entered may be used in the calculation of the probabilities as noted above. The various fields for this data entry screen are detailed above.

The tables referred to above are provided below.

TABLE 1
Baseline characteristics of the derivation and validation cohorts

| | Derivation | | Validation | |
|---|---|---|---|---|
| Variable | (n = 6,625) | | (n = 79,196) | |
| Demographic | | | | |
| Age, median (IQR), year | 59 | (67-75) | 67 | (60-75) |
| Age, n (%), year | | | | |
| ≤40 | 188 | (2.8) | 850 | (1.3) |
| 41-64 | 2.596 | (39.2) | 25.315 | (38.7) |
| 65-74 | 2.163 | (32.7) | 22.690 | (34.7) |
| 75-84 | 1.507 | (22.8) | 15.993 | (24.5) |
| ≥85 | 171 | (2.6) | 1.629 | (2.5%) |
| Female sex, n (%) | 1.851 | (27.9) | 15.993 | (24.5%) |
| Body mass index, n (%), kg/m² | | | | |
| <18.0 | 61 | (0.9) | 0 | |
| 18.1-24.9 | 1.779 | (26.9) | 17.059 | (26.1) |
| 25.0-29.9 | 2.577 | (38.9) | 25.769 | (39.4) |
| 30.0-34.9 | 1.446 | (21.8) | 14.896 | (22.3) |
| ≥35.0 | 762 | (11.5) | 7.686 | (11.8) |
| Comorbidities, n (%) | | | | |
| Hypertension | 4.855 | (73.3) | 56.521 | (86.4) |
| Myocardial infarction within | 1.407 | (21.2) | 16.185 | (24.7) |
| 30 days of surgery | | | | |
| Canadian Cardiovascular | | | | |
| Society classification | | | | |
| 0 | 2.751 | (41.5) | 12.620 | (19.3) |
| 1 | 492 | (7.4) | 5.583 | (8.5) |
| 2 | 1.070 | (16.2) | 10.574 | (16.2) |
| 3 | 1.100 | (16.6) | 10.963 | (16.8) |
| 4 | 1.212 | (18.3) | 25.670 | (39.2) |
| New York Heart | | | | |
| Association classification | | | | |
| 0 | 2.497 | (37.7) | 17.365 | (26.5) |
| 1 | 765 | (11.6) | 28.849 | (44.1) |
| 2 | 1.430 | (21.6) | 8.839 | (13.5) |
| 3 | 1.526 | (23.0) | 8.386 | (12.8) |
| 4 | 407 | (6.1) | 1.971 | (3.0) |
| Left ventricular ejection fraction | | | | |
| ≥50% | 4.914 | (74.2) | 44.844 | (68.6) |
| 35-49% | 1.009 | (15.2) | 14.228 | (21.8) |
| 20-35% | 474 | (7.2) | 5.421 | (8.3) |
| <20% | 228 | (3.4) | 917 | (1.4) |
| Atrial fibrillation | 1.117 | (16.9) | 4.704 | (7.2) |
| Endocarditis | 128 | (1.9) | 847 | (1.3) |
| Smoker (active or former) | 4.186 | (63.2) | 11.726 | (17.9) |
| Stroke | 748 | (11.3) | 6.759 | (10.3) |
| Peripheral arterial disease | 718 | (10.8) | 8.220 | (12.6) |
| Diabetes on medications | 1.761 | (26.6) | 20.652 | (31.6) |
| Anemia | 2.244 | (33.9) | 6.912 | (10.6) |
| Glomerular filtration rate, | | | | |
| mL min per 1.73 m² | | | | |
| ≥60 | 4.921 | (74.3) | 49.260 | (75.3) |
| 30-59 | 1.486 | (22.4) | 13.995 | (21.4) |
| <30 | 218 | (3.3) | 2.155 | (3.3) |
| Dialysis | 102 | (1.5) | 1.432 | (2.2) |
| Operative characteristics, n (%) | | | | |
| Emergent procedure | 531 | (8.0) | 9.930 | (15.2) |
| Preoperative cardiogenic shock | 244 | (3.7) | 2.700 | (4.1) |
| Redo sternotomy | 539 | (8.1) | 2.110 | (3.2) |
| Type of Surgery | | | | |
| CABG | 2.908 | (43.9) | 47.136 | (72.1) |
| Single valve | 1.176 | (17.8) | 9.245 | (14.1) |
| Valve(s) = CABG | 2.541 | (38.4) | 9.029 | (13.8) |

IQR = interquartile range;
CABG = coronary artery bypass grafting.

SUPPLEMENTARY TABLE 1: Covariates and their definitions.
These definitions are in keeping with definitions
employed by EuroSCORE¹ and or the STS database.²

| Covariates | Definition |
|---|---|
| Hypertension | A. BP > 140 mmHg systolic or > 90 mmHg diastolic in |
| | patients without diabetes or chronic kidney disease: |
| | or |
| | B. BP > 130 mmHg systolic or > 30 mmHg diastolic on at |
| | least two occasions in patients with diabetes or |
| | chronic kidney disease; |
| | C. History of hypertension treated with medication, |
| | diet, and/or exercise |
| Atrial | Documented history of paroxysmal or permanent atrial |
| fibrillation | fibrillation |
| Endocarditis | Endocarditis that is currently being treated with |
| | antibiotics |
| Peripheral | A. Claudication either with exertion or at rest: |
| arterial | B. Amputation for arterial vascular insufficiency: |
| disease | C. Vascular reconstruction, bypass surgery, or |
| | percutaneous intervention to the extremities: |
| | documented abdominal aneurysm with or without repair: |
| | D. Positive noninvasive test (ankle brachial index |
| | 0.9, ultrasound, MRA, CTA of >50% in any peripheral |
| | artery) or angiographic imaging |
| Diabetes on | Diabetes mellitus treated with oral hypoglycemic |
| medications | and/or insulin |
| Anemia | Defined by the World Health Organization³ (<130 g/L |
| | for men and <120 g/L for women), based on the |
| | hemoglobin concentration measured closest to the |
| | time of surgery. |
| Glomerular | Calculated using the Cockcroft-Gault formula⁴ |
| filtration rate | |
| Emergent | Surgery that must take place within 24 hours of |
| surgery | acute hospital admission |
| Preoperative | Requirement for inotropic support with evidence of |
| cardiogenic | end organ hypoperfusion or dysfunction or intraaortic |
| shock | balloon pump in situ before surgery |

References:
¹EuroSCORE. European System for Cardiac Operative Risk Evaluation. Available from URL: http://www.euroscore.org.
²The Society of Thoracic Surgeons National Database. Available from URL: http://www.euroscore.org
³Organization WH. Nutritional Anaemias: Report of a WHO Scientific Group. Geneva, Switzerland: World Health Organization. 1968.
⁴Cockcroft D W, Gault M H. Prediction of creatinine clearance from serum creatinine. Nephron. 1976: 16(1): 31-41.

TABLE 2
Multivariate analysis of patients with cardiac surgical intensive
care unit length of stay of ≤2 days vs. >2 days.

| | Model | | Wald | |
|---|---|---|---|---|
| Variable | β-Coefficient | OR (95% CI) | Chi-Square | P Value |
| Demographic | | | | |
| Age, year | | | | |
| ≤40 | NA | Reference | Reference | NA |
| 41-64 | −0.192 | 0.83 (0.56-1.23) | 0.92 | 0.339 |
| 65-74 | −0.404 | 0.67 (0.45-1.00) | 3.95 | 0.047 |
| 75-84 | −0.515 | 0.60 (0.40-0.90) | 6.09 | 0.014 |
| ≥85 | −0.795 | 0.46 (0.27-0.77) | 8.99 | 0.003 |
| Female sex | −0.169 | 0.84 (0.74-0.97) | 6.18 | 0.013 |
| Body mass index, kg/m² | | | | |
| <18.0 | −0.0408 | 0.96 (0.54-1.72) | 0.019 | 0.891 |
| 18.0-24.9 | NA | Reference | Reference | NA |
| 25.0-29.9 | −0.194 | 0.82 (0.71-0.96) | 6.12 | 0.013 |
| 30.0-34.9 | −0.461 | 0.63 (0.53-0.75) | 26.09 | <0.0001 |
| ≥35.0 | −0.703 | 0.50 (0.40-0.61) | 43.05 | <0.0001 |
| Comorbidities | | | | |
| CCS classification | | | | |
| 0 | NA | Reference | Reference | NA |
| 1 | −0.0087 | 0.99 (0.78-1.26) | 0.0051 | 0.94 |
| 2 | 0.147 | 1.16 (0.96-1.41) | 2.25 | 0.13 |
| 3 | 0.0341 | 1.04 (0.86-1.25) | 0.12 | 0.73 |
| 4 | −0.197 | 0.82 (0.67-1.00) | 3.72 | 0.05 |
| NYHA classification | | | | |
| 0 | NA | Reference | Reference | NA |
| 1 | −0.0656 | 0.94 (0.76-1.16) | 0.38 | 0.54 |
| 2 | −0.208 | 0.81 (0.69-0.96) | 5.94 | 0.01 |
| 3 | −0.538 | 0.59 (0.50-0.69) | 40.11 | <0.0001 |
| 4 | −1.288 | 0.28 (0.20-0.38) | 60.57 | <0.0001 |
| Left ventricular | | | | |
| ejection fraction | | | | |
| ≥50% | NA | Reference | Reference | NA |
| 35-49% | −0.386 | 0.68 (0.68-0.80) | 21.69 | <0.0001 |
| 20-35% | −1.043 | 0.35 (0.28-0.44) | 80.81 | <0.0001 |
| <20% | −1.479 | 0.23 (0.16-0.34) | 57.81 | <0.0001 |
| Atrial fibrillation | −0.302 | 0.74 (0.63-0.87) | 14.25 | 0.0002 |
| Endocarditis | −0.660 | 0.52 (0.33-0.81) | 8.66 | 0.003 |
| Stroke | −0.250 | 0.78 (0.65-0.93) | 7.40 | 0.007 |
| Peripheral arterial | −0.194 | 0.82 (0.69-0.99) | 4.28 | 0.04 |
| disease | | | | |
| Anemia | −0.373 | 0.69 (0.61-0.79) | 31.65 | <0.0001 |
| GFR, mL min 1.73 m² | | | | |
| ≥60 | NA | Reference | Reference | NA |
| 30-59 | −0.463 | 0.63 (0.54-0.74) | 32.45 | <0.0001 |
| <30 | −0.805 | 0.45 (0.32-0.63) | 21.72 | <0.0001 |
| Operative | | | | |
| characteristics | | | | |
| Emergent procedure | −0.914 | 0.40 (0.31-0.52) | 48.40 | <0.0001 |
| Preoperative | −1.218 | 0.30 (0.18-0.48) | 24.59 | <0.0001 |
| cardiogenic shock | | | | |
| Redo sternotomy | −0.539 | 0.58 (0.47-0.72) | 25.27 | <0.0001 |
| Type of Surgery | | | | |
| CABG | NA | Reference | Reference | NA |
| Single valve | 0.0131 | 1.01 (0.82-1.25) | 0.015 | 0.90 |
| Valve(s) = CABG | −0.785 | 0.46 (0.39-0.54) | 88.88 | <0.0001 |

OR = odds ratio;
CI = confidence interval;
MI = myocardial infarction;
CCS = Canadian Cardiovascular Society;
NYHA = New York Heart Association;
GFR = glomerular filtration rate;
CABG = coronary artery bypass grafting.

TABLE 3a
Observed versus predicted number of patients with a cardiac surgical intensive
care unit length of stay of ≤2 days in the validation cohort. The 95% confidence
intervals were obtained through 200 bootstraps with replacement.

| | Observed | | Predicted | | |
|---|---|---|---|---|---|
| Risk Quintile | Number | Rate (95% CI) | Number | Rate (95% CI) | OR (95% CI) |
| 1 (Low likelihood) | 6988 | 0.53 (0.52-0.54) | 5792.6 | 0.44 (0.44-0.45) | Reference |
| 2 (Low-moderate) | 9672 | 0.74 (0.73-0.75) | 9379.3 | 0.72 (0.72-0.72) | 2.47 (2.35-2.61) |
| 3 (Moderate) | 10614 | 0.81 (0.80-0.82) | 10659.1 | 0.82 (0.81-0.82) | 3.75 (3.55-3.97) |
| 4 (Moderate-high) | 11356 | 0.87 (0.86-0.87) | 11437.9 | 0.87 (0.87-0.87) | 5 58 (5.25-2.93) |
| 5 (High) | 11812 | 0.91 (0.90-0.91) | 11903.7 | 0.91 (0.91-0.91) | 8.44 (7.88-9.04) |

TABLE 3b
Observed versus predicted number of patients with a cardiac surgical intensive
care unit length of stay of ≥7 days in the validation cohort. The 95% confidence
intervals were obtained through 200 bootstraps with replacement.

| | Observed | | | Predicted | | | | |
|---|---|---|---|---|---|---|---|---|
| Risk Quintile | Number | Rate (95% CT) | | Number | Rate (95% CI) | | OR (95% CI) | |
| 1 (Low likelihood) | 111 | 0.008 | (0.007-0.01) | 128.5 | 0.009 | (0.009-0.009) | Reference | |
| 2 (Low-moderate) | 207 | 0.017 | (0.014-0.019) | 194.9 | 0.016 | (0.016-0.016) | 2.06 | (1.63-2.60) |
| 3 (Moderate) | 400 | 0.030 | (0.027-0.033) | 330.2 | 0.025 | (0.025-0.025) | 3.83 | (3.10-4.73) |
| 4 (Moderate-high) | 710 | 0.055 | (0.050-0.058) | 594.4 | 0.046 | (0.045-0.046) | 7.08 | (5.79-8.66) |
| 5 (High) | 1936 | 0.15 | (0.14-0.15) | 2253.2 | 0.17 | (0.17-0.18) | 21.26 | (17.53-25.78) |

TABLE 4
Baseline characteristics in those who died or had unplanned
cardiac hospitalizations and those who did not

| | No event | | Event | | Standardized |
|---|---|---|---|---|---|
| Variable | N = 59,342 | | N = 3,033 | | Differences |
| Demographics | | | | | |
| Age, Mean ± SD, y | 66.3 | (10.9) | 67.1 | (10.3) | 0.07 |
| Age, Median (IQR), y | 67 | (60-74) | 68 | (60-74) | 0.05 |
| Female sex, No. (%) | 15,626 | (26.3%) | 756 | (24.9%) | 0.03 |
| BMI, Mean ± SD, kg/m² | 28.95 | (5.56) | 28.56 | (5.38) | 0.07 |
| BMI, Median (IQR), kg/m² | 28 | (25-32) | 28 | (25-31) | 0.07 |
| Rural residence, No. (%) | 50,619 | (85.3%) | 2,619 | (86.4%) | 0.03 |
| Hospital type, No. (%) | | | | | |
| Community | 14,953 | (25.2%) | 379 | (12.5%) | 0.33 |
| Teaching | 44,389 | (74.8%) | 2,654 | (87.5%) | |
| Waitlisted during | 2,913 | (4.9%) | 770 | (25.4%) | 0.6 |
| inpatient encounter, | | | | | |
| No. (%) | | | | | |
| Comorbidities | | | | | |
| Hypertension, No. (%) | 49,488 | (83.4%) | 2,662 | (87.8%) | 0.12 |
| Atrial fibrillation, | 7,212 | (12.2%) | 418 | (13.8%) | 0.05 |
| No. (%) | | | | | |
| Recent MI, No. (%) | 2,119 | (3.6%) | 437 | (14.4%) | 0.39 |
| CCS classification, | | | | | |
| No. (%) | | | | | |
| 0 | 21,386 | (36.0%) | 575 | (19.0%) | 0.39 |
| 1 | 8,805 | (14.8%) | 483 | (15.9%) | 0.03 |
| 2 | 14,989 | (25.3%) | 569 | (18.8%) | 0.16 |
| 3 | 11,828 | (19.9%) | 603 | (19.9%) | 0 |
| 4 | 1,112 | (1.9%) | 163 | (5.4%) | 0.19 |
| Low-risk ACS | 858 | (1.4%) | 360 | (11.9%) | 0.43 |
| Intermediate-risk ACS | 339 | (0.6%) | 260 | (8.6%) | 0.39 |
| High-risk ACS | 25 | (0.0%) | 20 | (0.7%) | 0.1 |
| LM or LM equivalent | 18,319 | (30.9%) | 1,311 | (43.2%) | 0.26 |
| disease, No. (%) | | | | | |
| Proximal LAD disease, | 19,571 | (33.0%) | 1,346 | (44.4%) | 0.24 |
| No. (%) | | | | | |
| Previous PCI, No. (%) | 6,047 | (10.2%) | 375 | (12.4%) | 0.07 |
| Left ventricular | | | | | |
| ejection fraction, No. (%) | | | | | |
| ≥50% | 46,417 | (78.2%) | 2,108 | (69.5%) | 0.2 |
| 35-49% | 9,367 | (15.8%) | 611 | (20.1%) | 0.11 |
| 20-35% | 3,057 | (5.2%) | 273 | (9.0%) | 0.15 |
| <20% | 501 | (0.8%) | 41 | (1.4%) | 0.05 |
| NYHA classification, | | | | | |
| No. (%) | | | | | |
| 1 | 35,438 | (59.7%) | 2,038 | (67.2%) | 0.16 |
| 2 | 12,920 | (21.8%) | 409 | (13.5%) | 0.22 |
| 3 | 10,251 | (17.3%) | 460 | (15.2%) | 0.06 |
| 4 | 733 | (1.2%) | 126 | (4.2%) | 0.18 |
| Heart failure, No. (%) | 13,843 | (23.3%) | 968 | (31.9%) | 0.19 |
| Moderate-severe mitral | 6,951 | (11.7%) | 220 | (7.3%) | 0.15 |
| regurgitation, No. (%) | | | | | |
| Moderate-severe aortic | 2,278 | (3.8%) | 69 | (2.3%) | 0.09 |
| regurgitation, No. (%) | | | | | |
| Severe aortic stenosis, | 18,980 | (32.0%) | 687 | (22.7%) | 0.21 |
| No. (%) | | | | | |
| Endocarditis, No. (%) | | | | | |
| None | 58,852 | (99.2%) | 3,007 | (99.1%) | 0 |
| Acute | 154 | (0.3%) | 13 | (0.4%) | 0.03 |
| Subacute | 336 | (0.6%) | 13 | (0.4%) | 0.02 |
| Cerebrovascular disease, | 5,451 | (9.2%) | 314 | (10.4%) | 0.04 |
| No. (%) | | | | | |
| Peripheral arterial | 7,942 | (13.4%) | 412 | (13.6%) | 0.01 |
| disease, No. (%) | | | | | |
| Smoking status, No. (%) | | | | | |
| Never | 29,074 | (49.0%) | 1,448 | (47.7%) | 0.03 |
| Current | 9,129 | (15.4%) | 604 | (19.9%) | 0.12 |
| Former | 21,139 | (35.6%) | 981 | (32.3%) | 0.07 |
| COPD, No. (%) | 13,111 | (22.1%) | 795 | (26.2%) | 0.1 |
| Diabetes, No. (%) | 23,204 | (39.1%) | 1,435 | (47.3%) | 0.17 |
| Dyslipidemia, No. (%) | 39,600 | (66.7%) | 2,116 | (69.8%) | 0.07 |
| GFR, Mean ± SD, | 86.2 | (34.1) | 82.0 | (35.2) | 0.12 |
| mL/min/1.73 m² | | | | | |
| GFR, Median (IQR), | 82 | (62-105) | 79 | (58-103) | 0.11 |
| mL/min/1.73 m² | | | | | |
| Dialysis, No. (%) | 1,074 | (1.8%) | 90 | (3.0%) | 0.08 |
| Anemia, No. (%) | 2,344 | (3.9%) | 197 | (6.5%) | 0.11 |
| Liver disease, No. (%) | 561 | (0.9%) | 37 | (1.2%) | 0.03 |
| Alcohol abuse, No. (%) | 509 | (0.9%) | 43 | (1.4%) | 0.05 |
| Dementia, No. (%) | 656 | (1.1%) | 47 | (1.5%) | 0.04 |
| Depression, No. (%) | 415 | (0.7%) | 51 | (1.7%) | 0.09 |
| Psychosis, No. (%) | 70 | (0.1%) | 6 | (0.2%) | 0.02 |
| Primary cancer, No. (%) | 2,887 | (4.9%) | 150 | (4.9%) | 0 |
| Metastatic cancer, No. (%) | 287 | (0.5%) | 18 | (0.6%) | 0.02 |
| Operative characteristics | | | | | |
| Surgery type, No. (%) | | | | | |
| CABG | 30,481 | (51.4%) | 2,091 | (68.9%) | 0.36 |
| Valve | 18,781 | (31.6%) | 500 | (16.5%) | 0.36 |
| CABG + Valve | 7,518 | (12.7%) | 415 | (13.7%) | 0.03 |
| Thoracic Aorta | 2,562 | (4.3%) | 27 | (0.9%) | 0.22 |
| Redo-Sternotomy, No. (%) | 2,047 | (3.4%) | 131 | (4.3%) | 0.05 |
| Cardiogenic Shock, No. (%) | *52-59 | | *1-5 | | 0.01 |
| Operative priority, | | | | | |
| No. (%) | | | | | |
| Urgent | 20,296 | (34.2%) | 988 | (32.6%) | 0.03 |
| Semi-urgent | 11,180 | (18.8%) | 844 | (27.8%) | 0.21 |
| Elective | 27,866 | (47.0%) | 1,201 | (39.6%) | 0.15 |
| Recommend maximum wait | 43.7 | (34.2) | 41.3 | (31.0) | 0.07 |
| time, Mean ± SD, d | | | | | |
| Recommend maximum wait | 40 | (14-71) | 31 | (14-62) | 0.05 |
| time, Median (IQR), d | | | | | |
| Adherence to recommended | 31,126 | (52.5%) | 1,999 | (65.9%) | 0.28 |
| wait time**, No. (%) | | | | | |
| All-cause ED visits on | 0.1 | (0.5) | 0.3 | (0.6) | 0.28 |
| the waitlist, Mean ± SD | | | | | |
| All-cause ED visits on | 0 | (0-0) | 0 | (0-0) | 0.35 |
| the waitlist, Median (IQR) | | | | | |
| All-cause outpatient | 2.1 | (1.8) | 1.0 | (1.5) | 0.63 |
| physician visits on the | | | | | |
| waitlist, Mean ± SD | | | | | |
| All-cause outpatient | 2 | (1-3) | 1 | (0-2) | 0.75 |
| physician visits on the | | | | | |
| waitlist, Median (IQR) | | | | | |

*Data suppressed due to small cells
**Adherence is defined as adhering to procedure-specific wait times recommended by the Canadian Cardiovascular Society Access to Care Working Group (1).
Abbreviations:
SD = standard deviation;
IQR = interquartile range;
BMI = body mass index;
MI = myocardial infarction;
CCS = Canadian Cardiovascular Society;
ACS = acute coronary syndrome;
LM = left main;
LAD = left anterior descending;
PCI = percutaneous coronary intervention;
LVEF = left ventricular ejection fraction;
NYHA = New York Heart Association;
COPD = chronic obstructive pulmonary disease;
GFR = glomerular filtration rate;
CABG = coronary artery bypass grafting;
ED = emergency department

TABLE 5
Multivariate predictors of death or unplanned
cardiac hospitalization while on the waitlist

| | Model | | | |
|---|---|---|---|---|
| Variable | β-Coefficient | HR (95% CI) | | P-value |
| Demographics | | | | |
| GFR | −0.00220 | 1 | (1-1) | 0.003 |
| BMI | −0.01390 | 0.99 | (0.98-1) | 0.004 |
| Teaching vs. | 0.58030 | 1.79 | (1.56-2.05) | <.0001 |
| community hospital | | | | |
| Waitlisted during | 1.62000 | 5.05 | (4.48-5.7) | <.0001 |
| inpatient encounter | | | | |
| Rural Residence | −0.15610 | 0.86 | (0.75-0.97) | 0.02 |
| Comorbidities | | | | |
| CCS classification | | | | |
| 0 | NA | Reference | | NA |
| 1 | 0.46610 | 1.59 | (1.36-1.87) | <.0001 |
| 2 | 0.17870 | 1.2 | (1.02-1.4) | 0.03 |
| 3 | 0.40100 | 1.49 | (1.27-1.75) | <.0001 |
| 4 | 1.00830 | 2.74 | (2.18-3.45) | <.0001 |
| Low-risk ACS | 1.79390 | 6.01 | (4.96-7.29) | <.0001 |
| Intermediate-risk ACS | 2.12500 | 8.37 | (6.68-10.49) | <.0001 |
| High-risk ACS | 2.08940 | 8.08 | (4.81-13.56) | <.0001 |
| Recent MI | −0.17960 | 0.84 | (0.72-0.97) | 0.02 |
| NYHA classification | | | | |
| 1 | NA | Reference | | NA |
| 2 | −0.32440 | 0.72 | (0.62-0.84) | <.0001 |
| 3 | −0.06690 | 0.94 | (0.8-1.09) | 0.4 |
| 4 | 0.57800 | 1.78 | (1.4-2.26) | <.0001 |
| Heart failure | 0.33430 | 1.4 | (1.25-1.56) | <.0001 |
| Atrial Fibrillation | 0.23010 | 1.26 | (1.1-1.44) | 0.0007 |
| Diabetes | 0.12470 | 1.13 | (1.03-1.24) | 0.008 |
| Proximal LAD | 0.10790 | 1.11 | (1.01-1.23) | 0.03 |
| Aortic Stenosis | 0.24900 | 1.28 | (1.08-1.52) | 0.004 |
| Endocarditis | | | | |
| None | NA | Reference | | NA |
| Acute | 0.77580 | 2.17 | (1.16-4.08) | 0.02 |
| Subacute | −0.24120 | 0.79 | (0.37-1.66) | 0.5 |
| Operative characteristics | | | | |
| Surgery type | | | | |
| CABG | NA | Reference | | NA |
| Valve | −1.09820 | 0.33 | (0.27-0.41) | <.0001 |
| CABG + Valve | −0.49070 | 0.61 | (0.49-0.76) | <.0001 |
| Thoracic Aorta | −1.81340 | 0.16 | (0.1-0.27) | <.0001 |
| Operative priority | | | | |
| Urgent | 0.37080 | 1.45 | (1.24-1.69) | <.0001 |
| Semi-urgent | 0.40440 | 1.5 | (1.34-1.68) | <.0001 |

| | | | |
|---|---|---|---|
| Elective | NA | Reference | NA |

Abbreviations:
BMI = body mass index;
CCS = Canadian Cardiovascular Society;
ACS = acute coronary syndrome;
MI = myocardial infarction;
NYHA = New York Heart Association;
LAD = left anterior descending;
GFR = glomerular filtration rate;
CABG = coronary artery bypass grafting

It should be clear that various aspects of the present invention may be implemented as software modules in an overall software system. As such, the present invention may thus take the form of computer-executable instructions that, when executed, implements various software modules with predefined functions.

Embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network. Various embodiments of the differing aspects of the invention may also take the form of computer programs that are available for use and/or download from online repositories. Similarly, other embodiments may take the form of computer software that is stored and/or executable and/or hosted from an online repository or from an online server.

Embodiments of the invention may be implemented in any conventional computer programming language. For example, preferred embodiments may be implemented in a procedural programming language (e.g., “C” or “Go”) or an object-oriented language (e.g., “C++”, “java”, “javascript”, “PHP”, “PYTHON” or “C#”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for managing an electronic calendaring system, said system comprising:
a server configured to produce at least one schedule element detailing at least one medical procedure for a specific patient at a healthcare facility, and said server being configured to:
receive patient data, said patient data relating to said specific patient;
implement a length of stay (LOS) model, said LOS model producing at least one LOS probability related to said specific patient's projected LOS at said healthcare facility, wherein said at least one LOS probability is based on said patient data;
implement a waitlist model for calculating a waitlist probability based on said patient data, said waitlist probability being a probability of an emergent event of said specific patient while said specific patient remains on a waiting list for said at least one medical procedure;
implement a scheduling model, said scheduling model being a trained AI model configured to receive said at least one LOS probability, said waitlist probability, and current occupancy data of said healthcare facility as inputs, and said scheduling model being configured to output said at least one schedule element;
receive said at least one schedule element as output from said scheduling model; and
automatically enter said schedule element in said calendaring system,
wherein said at least one schedule element is configured for entry in said electronic calendaring system,
wherein said at least one schedule element has a specific set of components,
wherein said components of said schedule element comprise: a type of a medical procedure; a date for said medical procedure; a time for said medical procedure; a location for said medical procedure; a list of resources required for said medical procedure; and a list of invitees for said medical procedure, and
wherein values of said components are automatically determined by said scheduling model.

2. The system according to claim 1, wherein said scheduling model predicts a cumulative distribution of occupancy at said healthcare facility over a predetermined time period.

3. The system according to claim 2, wherein said scheduling model minimizes said cumulative distribution when determining said values of said components.

4. The system according to claim 1, wherein entering said schedule element in said calendaring system causes at least one further change in said calendaring system, and wherein specifics of said further change are also determined by said scheduling model.

5. The system according to claim 1, wherein said server is further configured to inspect said schedule element before entering said schedule element in said calendaring system, wherein inspection of said schedule element comprises comparing said values of said components of said schedule element to values of components of other calendar entries already in said calendaring system,
such that, when values of a subset of said components of said schedule element match values of corresponding components of a specific entry of said other entries, said server identifies a conflict between said schedule element and said specific entry.

6. The system according to claim 5, wherein, when said conflict is identified, said server feeds back said at least one LOS probability and said waitlist probability of said specific patient to said scheduling model, along with input data for said specific entry, to thereby produce at least one of a revised schedule element and a revised specific entry, said revised schedule element having at least one component value that differs from a component value of said schedule element and said revised specific entry having at least one component value that differs from a component value of said specific entry.

7. The system according to claim 6, wherein said scheduling model produces both a revised schedule element and a revised specific entry.

8. The system according to claim 1, wherein said scheduling model also takes existing entries in said calendaring system as inputs, and wherein said values of components of said schedule element do not conflict with components of said existing entries.

9. The system according to claim 1, wherein said scheduling model generates a schedule element for all patients at said healthcare facility at a specific time, thereby generating a full schedule for medical procedures at said healthcare facility.

10. The system according to claim 1, wherein said scheduling model is trained on population-wide data and on historical data from said healthcare facility.

11. The system according to claim 1, wherein said emergent event is at least one of an unplanned hospitalization of said at least one patient and a mortality of said at least one patient.

12. The system according to claim 1, wherein at least one of said LOS model and said waitlist model is a trained AI model.

13. The system according to claim 12, wherein both of said LOS model and said waitlist model are trained AI models.

14. The system according to claim 1, wherein said list of invitees comprises at least one medical professional.

15. The system according to claim 15, wherein said at least one medical professional is identified by said scheduling model by assessment of said type of said medical procedure and of personnel records of said healthcare facility, such that said at least one medical professional has a suitable skill level for said medical procedure.

16. The system according to claim 1, wherein said server is further configured to transmit said schedule element to at least one person on said list of invitees before entering said schedule element in said calendaring system.

17. The system according to claim 2, wherein said cumulative distribution of occupancy at said healthcare facility is a predicted distribution of occupancy of a specific subset of said healthcare facility.

18. The system according to claim 17, wherein said specific subunit of said healthcare facility is an Intensive Care Unit (ICU).

19. The system according to claim 2, wherein said cumulative distribution of occupancy at said healthcare facility comprises a predicted distribution of occupancy in an Intensive Care Unit (ICU) and a predicted distribution of occupancy in said healthcare facility.

20. The system according to claim 1, wherein said at least one patient is designated a high-risk patient when said waitlist probability of said at least one patient is high and wherein said scheduling model is trained to prioritize medical procedures such that high-risk patients are scheduled for near-term medical procedures.
